(19) United States
(12) Patent Application Publication
(10) Pub. No.: US 2011/0272320 A1
(43) Pub. Date: Nov. 10, 2011

(54) Aerosol Shave Composition Comprising A Hydrophobical Agent Forming At Least One Microdroplet

(76) Inventors: Ali Abdelaziz Alwattari, West Chester, OH (US); Marcia J. Holden, Harrison, OH (US); George Endel Deckner, Cincinnati, OH (US); Theresa Louise Johnson, South Lebanon, OH (US); Vickie Lynn Cornelius, Loveland, OH (US)

(21) Appl. No.: 13/101,104

(22) Filed: May 4, 2011

Related U.S. Application Data
(60) Provisional application No. 61/332,044, filed on May 6, 2010.

(51) Int. Cl. B65D 85/84; A61K 8/89; A61K 8/898; A61K 8/81; A61K 8/73; A61K 8/72; A61K 8/88; A61K 8/04; A61Q 9/02

(52) U.S. Cl. 206/524.1; 424/73; 424/70.12; 424/70.122; 424/70.13; 424/70.11; 424/70.17; 424/70.16; 424/70.31

(57) ABSTRACT

An aerosol shave composition, preferably a post foaming shave gel, comprising a hydrophobic agent, such as a silicone, in the form of a microdroplet having a particle size from about 0.15 microns to about 10 microns.

### CROSS REFERENCE TO RELATED APPLICATIONS

This patent claims priority to U.S. Provisional Application No. 61/332,044 filed May 6, 2010.

### BACKGROUND OF THE INVENTION

Post-foaming shave gels are now well-known. See, e.g., U.S. Pat. Nos. 5,326,556 and 5,500,211. Various attempts have been made to increase the lubricity of shaving compositions. The addition of various polymers into personal care compositions is known. See e.g. U.S. Patent Publ. No. 2007/0207106; U.S. Pat. Nos. 5,902,574 and 5,262,154. Further, in some cases a lubricious water soluble polymer such as polyethylene oxide or polyvinylpyrrolidone has been added. See, e.g., U.S. Pat. Nos. 5,560,859 and 5,858,343. In other cases, water insoluble particles have been added, including water insoluble polymer particles, such as polytetrafluoroethylene, polyethylene, or polyamide (nylon) particles, and water insoluble inorganic particles such as titanium dioxide or glass beads. See, e.g., U.S. Pat. Nos. 5,587,156 and 4,155,870. Various other shave gels have been disclosed. See, e.g., U.S. Patent Publ. No. 2006/0257349, 2006/0257350 and 2005/0175575 and U.S. Pat. Nos. 5,500,211 and 6,352,689, and WO Publication 2010/009989.

To improve the conventional shaving process, manufacturers offer various types of shave oils which include the shave oils manufactured by King of Shaves. These shave oils typically include various forms of silicone or mineral oils, and are described for use in substitution of foaming shave preparations, or before or after application of foaming shaving preparations. The addition of these shave oil ingredients into a fully formulated post foaming gel is believed to be desirable to provide the lubrication benefits of the shave oil, while maintaining the skin comfort of a foaming preparation.

It has been reported that certain polyorganosiloxane microemulsions having average particle size of less than 0.14 microns are suitable for introduction into aerosol or post foaming gels. See U.S. Pat. No. 5,523,081. Formulating post foaming shave gels containing polyorganosiloxane microemulsions having such specific average particle size limitations can be difficult to manufacture due to the intense processing constraints needed to make such small particles. As such, there remains a need for a new post foaming gel which can provide enhanced lubrication yet maintain skin comfort and foaming capabilities while being sufficiently manufacturable on a commercial scale. The present invention addresses one or more of these needs.

### SUMMARY OF THE INVENTION

One aspect of this invention relates to a personal care composition which is in the form of an aerosol product, preferably a post foaming gel or a shaving foam, said composition comprising: from about 2% to about 25% of a water dispersible surface active agent; from about 40% to about 95% of a carrier comprising water; from about 1% to about 6% of a volatile post-foaming agent; and from about 0.01 to about 15% of a hydrophobic agent, wherein said hydrophobic agent forms at least one microdroplet having a particle size from about 0.15 microns to about 10 microns.

### DETAILED DESCRIPTION OF THE INVENTION

The term “fatty”, as used herein, means a hydrocarbon chain having 12-22 carbon atoms (C12-22), preferably 14-18 carbon atoms (C14-18). The chain may be straight or branched and may be saturated or unsaturated (typically one or two double bonds in the chain). The term “water dispersible”, as used herein, means that a substance is either substantially dispersible or soluble in water.

The personal care composition of the present invention is suitable for use as a hair removal preparation, such as a post-foaming shave gel composition. In one embodiment the composition comprises from about 0.005% to about 3% of a cationic polysaccharide, wherein said cationic polysaccharide is hydrophobically modified; about 2% to about 25%, preferably about 5% to about 20%, of a water dispersible surface active agent, from about 60% to about 93%, or from about 70% to about 85% of a carrier, such as water; and a lubricant. The lubricant can comprise preferably about 0.01% to about 1%, lubricious water soluble polymer, about 0.01% to about 5%, preferably about 0.1% to about 2%, water insoluble particles, and about 0.0005% to about 3%, preferably about 0.001% to about 0.5%, hydrogel-forming polymer, by weight of the composition. Preferably, the composition is in the form of a post-foaming shave gel and will additionally include about 1% to about 6%, preferably about 2% to about 5%, volatile post-foaming agent.

In one embodiment, personal care compositions of the present invention have a viscosity of from about 85 to about 3000 cps, in an alternate embodiment from about 185 to about 2500 cps, in an alternate embodiment from about 190 to about 2000 cps, in an alternate embodiment from about 200 to about 1900 cps as measured by a commercial dip probe rheometer, such as the Hydramotion Viscolite 700 Model VL700s (solid state insertion viscosity meter). The viscosity in the current invention is determined by submerging the testing probe into 200 milliliters of the formulation, activate the probe, and record the result after 60 seconds of stabilization time.

Without wishing to be bound by theory, it is believed that personal care compositions of the present invention having increased viscosity provide for superior protection because they are more effective at high shear rates. Moreover, the high viscosities of the present invention surprisingly occur while achieving desirable lubrication benefit which can be shown by coefficient of friction measurements.

In one embodiment, the aerosol shave composition is a substantially homogeneous mixture of its constituents. Homogenous, as defined herein, means that the composition has a uniform mixture throughout and no distinct regions can be observed with the naked eye. Those of ordinary skill in the art will understand that the hydrophobic agent can form microdroplets. These microdroplets, however should not create a visible phase separation from the rest of the composition. It has importantly been found that the present invention allows for the benefits delivered by conventional shave preps and pre-shave oils and other treatments, to be conveniently delivered in a single composition which can provide enhanced lubrication yet maintain skin comfort and foaming capabilities in a single composition which does not need to be shaken or otherwise mixed by the user prior to dispensing from the container. In one embodiment, the composition can further be contained in a single container having a single compartment.

### 1. Hydrophobic Agent

The aerosol shave composition of the present invention comprises a hydrophobic agent. The level of the hydrophobic agent can be from about 0.01% to about 15% by weight of said aerosol shave composition, preferably from about 0.1% to about 10%, more preferably from about 0.2% to about 5%, even more preferably from about 0.5% to about 2%.

Non-limiting examples of hydrophobic agents which can be used in accordance with the present invention comprises at least one of a silicon polymer, an emollient oil, a mineral oil, water soluble vitamins (such as vitamin E and vitamin A), oil soluble fragrances, oil soluble colorants, and any oil soluble sensates, anhydrous polyols, and mixtures thereof. In one embodiment, the silicon polymer comprises any member of the dimethicone family, such as at least one of an organosiloxane, an amino-functional siloxane, and combinations thereof. In one embodiment, the organosiloxane comprises at least one of a dimethicone, a trimethylsiloxane, a polydimethylsiloxane, a silicone elastomer, and combinations thereof. Examples of suitable organosiloxanes include the polyorganosiloxanes disclosed in U.S. Pat. Nos. 6,096,697, 5,523,081, 4,749,732, 4,620,878, 5,015,682 (carboxyglycol ether and carboxy glycol ester functional polysiloxanes; EP 0268982 (polydiorganosiloxanes), and aminofunctional polydiorganosiloxanes as disclosed in EP 0514934. One non-limiting example of a suitable amino functional silicone is the dimethicone is family of Copolymer of Acrylamide (AM) and TRIQUAT. Nonlimiting examples of suitable emollient and mineral oils include any which are commercially available and use for skin care or cosmetic purposes.

In one embodiment the silicon polymer is a silicone having a viscosity of from about 20 to about 2,000,000 centistokes, preferably from about 1,000 to about 1,800,000 centistokes, preferably from about 3,000 to about 1,500,000 centistokes, preferably from about 10,000 to about 1,000,000, preferably from about 30,000 to about 60,000 centistokes, at 25° C. The viscosity can be measured by means of a glass capillary viscometer as set forth in Dow Corning Corporate Test Method CTM0004, Jul. 20, 1970. The silicon can also be a silicone oil which is a flowable silicone materials with a viscosity of less than 1,000,000 centistokes, preferably between about 5 and 1,000,000 centistokes, more preferably between about 10 and about 600,000 centistokes, more preferably between about 10 and about 500,000 centistokes, most preferably between 10 and 300,000 centistokes at 25° C. Suitable silicone oils include polyalkyl siloxanes, polyaryl siloxanes, polyalkylaryl siloxanes, polyether siloxane copolymers, and mixtures thereof. Other insoluble, nonvolatile silicone fluids having conditioning properties can also be used. Suitable silicone oils for use in the composition include polyalkyl or polyaryl siloxanes which conform to following formula:

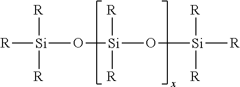

where R is aliphatic, preferably alkyl or alkenyl, or aryl, R can be substituted or unsubstituted, and x is an integer from 1 to about 8,000. Suitable unsubstituted R groups include alkoxy, aryloxy, alkaryl, arylalkyl, arylalkenyl, alkamino, and ether-substituted, hydroxyl-substituted, and halogensubstituted aliphatic and aryl groups. Suitable R groups also include cationic amines and quaternary ammonium groups. The aliphatic or aryl groups substituted on the siloxane chain may have any structure as long as the resulting silicones remain fluid at room temperature, are hydrophobic, are neither irritating, toxic nor otherwise harmful when applied to the hair or skin, are compatible with the other components of the herein described personal cleansing compositions, are chemically stable under normal use and storage conditions, are insoluble in the compositions of the present invention, and are capable of being deposited on and, of conditioning, and lubricating the hair and skin.

In one embodiment, the ratio of hydrophobic agent to volatile post foaming agent is from about 1:3 to about 3:1, preferably from about 1:2 to about 2:1, even more preferably about 1:1, even more preferably about 2.5:2.85.

Without intending to be bound by theory, it is believed that the hydrophobic agent of the present invention provides for the desired lubrication benefit previously observed when users combined a shave oil with a conventional shave foam which is a two step shaving preparation process. It is believed that the addition of the specific hydrophobic agent of the present invention, via the form of microdroplets, allows the present aerosol shave gel (i.e. the post foaming gel) to provide the lubrication benefits desired from a shave oil while maintaining the desirable foaming matrix to give cushion and comfort during shaving.

Specific examples of suitable hydrophobic agents include: polydimethylsiloxane (PDMS) having a viscosity from about 1 cs up to about 300,000 cs, commercially available emulsions of dimethicone (which can be pre-made by supplier, such as HMW 2220 from Dow Corning with an internal phase (dimethicone) viscosity of greater than about 1 million cs); Amino and Quat-functional silicones (which can include Terminal Amino Silicone with a viscosity of about 10000 cs and Abil T Quat from Evonik); hydrophilically-modified silicones (such as silicone polyethers available across a wide range of EO/PO substitutions) and dimethiconol from suppliers such as Momentive, Dow Corning, and Shin Etsu.

In one embodiment, the composition comprises more than one hydrophobic agent. For example the composition can comprise amino functional silicones and quat-functional silicones with other hydrophilic functionalities e.g. Waro Silicone Quats (Momentive) and SiLC (Silicone Low Cost) which are aminosilicones (terminal and pendant) with hydrophilic groups added via glycidol or PPG groups), as well as sugar functional silicones available from Dow Corning and Wacker—these generally include amine groups as well to link the silicone backbone to the saccharide groups. In one embodiment, the functionalized silicone can be a phenyl silicone.

### 2. Microdroplets

Microdroplets, as defined herein means a fluid particle having the particle size range and/or the average particle size as defined herein. As explained in the Background, herein, it has been reported that the addition of certain polyorganosiloxane microemulsions having average particle size of less than 0.14 microns are desirable for introduction into aerosol or post foaming gels. See U.S. Pat. No. 5,523,081. The present invention, however, has found that microdroplets of the present invention are particularly desirable despite the teachings of U.S. Pat. No. 5,523,081.

Without intending to be bound by theory, it is believed that increasing the sizes of the hydrophobic agent beyond the sizes disclosed in U.S. Pat. No. 5,523,081 provides for the desired lubrication benefits yet maintaining the desired compositional rheology (i.e. thickness and viscosity) and foaming forming ability of the aerosol shave composition of the present invention.

For example contrary to what some may believe, providing the microdroplets into the present composition has not been found to be subject to a common potential problem associated with the presence of polymer compounds in finished consumer product compositions containing other hydrophobic ingredients i.e., tackiness and stringiness. Not wishing to be bound by theory, it is thought that by providing a dispersion of the microdroplets, the hydrophobic agent is less likely to interact with other functional ingredients (such as those commonly included for lathering, lubrication, and/or spreading, including but not limited to: propellants, polymers, and soap) allows the hydrophobic agent to deliver the desired lubrication and sensory benefits previously only obtainable through a multi-step multi product shaving process. Importantly, now, we are able to provide some of the benefits of the hydrophobic agent with the shaving composition.

Also, it is believed that the majority of hydrophobic and low surface tension materials typically reduce and decrease the lather and foam forming performance of conventional foaming compositions. The present invention provides desirable lubrication benefits while maintaining the desired foam and lather performance. Without intending to be bound by theory, it has been found that despite the microdroplets having sizes larger than those described in the past, the present invention provides the desired compositional properties while providing the desired shave benefits.

Without intending to be bound by theory, it is believed that a composition comprising a hydrophobic agent of the present chemical make up and physical dimensions can be present in such a composition but not appear as a separate phase by the naked eye. In one embodiment, the hydrophobic agent forms a discontinuous phase and the other components of the composition can form a continuous phase with any aqueous components. Those of skill in the art will understand that although discreet particles can be viewed using tools such as a microscope, the composition will appear as a single substantially homogenous mixture of uniform appearance to when it is in the gel (pre-foamed) state. Without intending to be bound by theory, it is believed that the present composition can deliver 2 in 1 type benefits previously obtained by using a pre-shave oil and a foaming shave preparation. By providing multiple shaving benefits in a single composition the process of shaving can be simplified by obfuscating the need for the extra step of applying a shave oil. Further, it is believed that by adding the hydrophobic agent into the present composition, users of conventional shaving preps can now benefit from the presence of the hydrophobic agents which previously involved extra pre-shaving steps, such as application of an oil before applying a shave prep.
a. Particle Size Range

In one embodiment, the hydrophobic agent present in the aerosol shave gel forms at least one microdroplet having a particle size from about 0.15 microns to about 10 microns, preferably from about 0.5 microns to about 5 microns, preferably from about 1.5 microns to about 3 microns, more preferably about 2 microns.

In another embodiment, the microdroplet can have a particle size from about 0.5 micron to about 50 microns, preferably from about 2 microns to about 10 micron, or from about 2 microns to about 5 microns. It is believed that this second set of ranges for microdroplets can occur when the composition is allowed to rest after manufacturing. Depending on the specific formulation, a certain amount of relatively smaller microdroplets may combine to form relatively larger microdroplets, such as those described in this paragraph. Without intending to be bound by theory, it is believed that larger microdroplets can deliver increased benefits such as skin moisturization since larger droplets allow more of the agent to be released onto skin during application.
b. Average Particle Size

In another embodiment, the hydrophobic agent in the aerosol shave gel forms a plurality of microdroplets comprising an average particle size of from about 0.5 microns to about 3 microns, preferably from about 1.5 about to about 2.5 microns, more preferably from about 1.9 microns to about 2.4 microns, even more preferably about 2 microns.

As used herein, average particle size is in reference to the largest outer linear dimension of particles formed by the hydrophobic agent and is determined by the Dynamic Light Scattering Method as defined herein.

### Dynamic Light Scattering Method:

The Dynamic Light Scattering Method measures the average diameter of the lamellar vesicles by light scattering data techniques, which is an intensity-weighted average diameter. One suitable machine to determine the average diameter is a Brookhaven 90Plus Nanoparticle Size Analyzer. A dilute suspension with concentration ranging from 0.001% to 1% v/v of the sample being tested using a suitable wetting and/or dispersing agent (i.e. water) is prepared. A 10 mL sample of the suspension is placed into a sample cell and measurements are recorded providing average particle size.
c. Additives Dissolved into the Hydrophobic Agent

One or more of the adjunct ingredients described below in section titled “Other Adjunct Ingredients” can be used as an additive at least partially dissolved into the hydrophobic agent. In one embodiment, all of the additive can be dissolved into the hydrophobic agent before the agent is turned into a microdroplet. Those of skill in the art will also understand that the additive(s) can be in a neat form and not dissolved into the hydrophobic agent. In one embodiment, the additive dissolved in the hydrophobic agent is any suitable skin care composition which is dissolvable in a hydrophobic agent, such as silicone. In one embodiment, the additives is an aesthetic component (e.g., fragrances, pigments, and colorings/colorants) essential oils, skin sensates, excipients and/or astringents (e.g., clove oil, menthol, camphor, eucalyptus oil, eugenol, menthyl lactate, witch hazel distillate, clobetasol). Additional additives can be selected from the sensates, excipients or cooling agents described below. Non-limiting examples of suitable additives include: menthol dissolved in the silicone which can provide a more intense burst and control of release profile, fragrance added to silicone which can give an all day after shave smell, and oil soluble dyes or colorants which can provide visual aesthetics to the composition.

In one embodiment, the additives can be provided at a level of from about 1% to about 99% by weight of the hydrophobic agent. The additives can also be added to the hydrophobic agent prior to any processing steps which are used to make the hydrophobic agent into the microdroplets. In another embodiment, where the additive is in a neat arrangement (as a direct add into the main chassis of the composition, they can be added with other adjunct ingredients during processing. Without intending to be bound by theory, it is believed that the presence of additives in the hydrophobic agent, and in a neat product form can provide benefits such as a faster cooling feel, increased or prolonged sensation or fragrance, and so forth.

### 3. Microdroplet Premix

The hydrophobic agent of the present invention is preferably formed into the microdroplets via an emulsion premix. A microdroplet premix, as defined herein, means that the hydrophobic agent is mixed with a carrier, such as water. In one embodiment, the microdroplet premix comprises at least about 50% of a hydrophobic agent and up to about 50% of a carrier such as an aqueous material. In one embodiment, the microdroplet premix is an oil-in-water emulsion. In another embodiment, the microdroplet premix is a water-in-oil emulsion. In another embodiment, the carrier further comprises one or more of: organic cosolvents, glycerin, diglycerin, sorbitol, butylene glycol, propylene glycol, PEG 4, and mixtures thereof.

In one embodiment, the ratio of hydrophobic agent to carrier (optionally with the emulsifier) is from about 99.8:2 to about 1:99, preferably from about 50:50 to about 95:5. It is believed that by providing a microdroplet premix having a relatively large amount of hydrophobic agent in a carrier is particularly preferable to achieve the microdroplets when the hydrophobic agent is added to the other ingredients of the composition. The carrier may also contain water dispersible surface active agents, water stabilizing particles (e.g. zeolites), thickening polymers, nano-latexes in hydrophilic liquid, surface tension reducing polymers (like cyclodextrin), microgels such as pemulen, natrosol plus 330 or other polymeric stabilizers such as lauryl dimethicone/copolyol crosspolymer, or mixtures thereof.

Importantly, it has been found that when a commercially available shave oil is merely added directly into an aerosol shave gel formulation, then mixed, the shave oils form silicone particles having particle sizes in the range of 10 microns or larger. These mixtures, however, were found to be undesirably runny and failed to make the desired foam when tested. Also a high degree of shearing is needed to introduce viscous hydrophobic agents (such as oils) into a carrier (water) to form a continuous gel formulation. This can impeded the ability of the water dispersible surface active and propellants to ultimately lather and form foams during use. However, the premix step of the present invention enables gentle incorporation of the hydrophobic agent into the formula while maintaining homogeneity and integrity of the composition. Without intending to be bound by theory, it is believed that failing to form an microdroplet premix may cause hydrophobic agents either to form relatively large droplets or to get entrapped inside soap micelles and not be free to lubricate the hair and skin thoroughly, as well as interacting with the volatile foaming agents which are added to the mixture, thereby impacting the viscosity of the product as it is dispensed out of the aerosol can during use.

The microdroplet premix can also include one or more emulsifiers. In one embodiment, the emulsifier comprises a surfactant, a water-soluble emulsification polymer, or a mixture thereof. In one embodiment, the emulsifier comprises a water-soluble emulsification polymer having a molecular weight of at least about 500 Daltons, or at least about 3000 Daltons, or at least about 9000 Daltons, or at least about 10,000 Daltons. An upper limit is defined by processability such as if the weight is above 100,000 Daltons the carrier material may be too water insoluble and difficult to fill with microdroplets but this has not yet been encountered as a problem in practice.

Non-limiting examples of suitable emulsifiers include alkyl glucosides such as decyl glucoside and lauryl glucoside, laureth 7, sisterna L70C, ECTD 3NEX, plantaren 2000, sucrose cocoate, polyglycerol 10 laurate, laureth 6 carboxylate, and mixtures thereof. In another embodiment, the emulsifier comprises at least one non-alkoxylated water-soluble emulsification polymer. Examples of suitable non-alkoxylated water-soluble emulsification polymers are described in U.S. Patent Publs. 2005/0031659, 2005/0031568, and 2005/0032916, each to Deckner. Without intending to be bound by theory, it is believed that the use of emulsifiers in forming the microdroplet premix is particularly useful in that they help in the formation of the microdroplets.

Without wishing to be bound by theory, it is believed that personal care compositions of the present invention having increased viscosity provide for superior protection because they are more effective under high shear (such as when rubbed from the hand or another applicator onto skin) Moreover, the high viscosities of the present invention surprisingly occur while creating improved lubrication benefits as measured by the In-Shave Lubrication Method, as defined herein.

In one embodiment, the microdroplet premix forms the microdroplet as described herein. In one embodiment, the hydrophobic agent in the microdroplet premix and the hydrophobic agent forming said at least one microdroplet in the aerosol shave composition have the same particle size range and/or average particle size as described with respect to the microdroplets.

In one embodiment, the microdroplet premix is free or essentially free of electrolyte. As used herein, essentially free of a component means that no amount of that component is deliberately incorporated into the composition. Residual or carry over amounts of said components may exist as long as no amount is deliberately added, preferably no more than 0.01% or 0.001% by weight.

In another embodiment, the microdroplet premix comprises a ratio hydrophobic agent to emulsifier of from about 50:50 up to about 95:5. Preferably, after the microdroplet premix is formed, it is then added and mixed into the shave gel concentrate (i.e. remaining ingredients) prior to gassing with the volatile post foaming agent and allowing the gel to set.

In one embodiment, the premix is formed in a single mixing step. In another embodiment, premix can be formed in multiple steps. In one embodiment, the method of making the premix comprises at least three steps. 1) Forming a carrier, which can comprise combining one or more emulsifiers, one or more additives, or mixtures thereof with water and mixing. 2) Adding discrete batches of 2-3% of the total weight of the hydrophobic agent are titrated sequentially into the aqueous phase accompanied by gentle mixing to obtain a uniform consistency prior to addition of the following batch. This is continued until about 20% of the total weight of hydrophobic agent has been added. As this point the remainder of the oil may be added more rapidly and in a continuous fashion with more vigorous mixing until a uniform emulsion comprising all of the hydrophobic agent is obtained. 3) Mixing is continued until a uniform consistency is obtained wherein the majority, or at least 75%, or at least 90%, or at least 95%, or substantially all of the hydrophobic agent is in the form of microdroplets.

In one embodiment, steps 1 and 2 can be performed simultaneously, in that the carrier can be formed while adding the discrete batches of the hydrophobic agent. As such, a step of premixing the carrier ingredients may be unnecessary.

In one embodiment, the premix is formed by mixing the hydrophobic agent with the carrier and optionally with the emulsifier, additives or mixtures thereof. This step of forming the premix can be under low shear such as by hand mixing or by a conventional mixer which can make emulsions. In one embodiment the forming step is performed for at least 5 minutes, or for a period of from about 5 minutes to about 60 minutes, preferably about 30 minutes.

Where the premix is made on a lab or bench scale, a medium shear conventional mixer can be used such as a Kitchen Aid® Ultra Power Mixer with a paddle attachment. The mixer can be set on any setting to form the carrier if multiple ingredients are present in the carrier. When the hydrophobic agent is added to the carrier, the mixer can be run at a setting of 2, 3 or 4, depending on the viscosity of the ingredients. The mixing can be performed until a desirable premix is obtained. Another suitable mixer for forming the premix includes a Cito Unguator which can be run at a setting of 5. Those of skill in the art will understand that other more industrial mixers can be used where the premix is formed on a commercial scale.

The premix can also be formed at higher shears such as by using a Speed Mixer DAC 800 FVZ with an RPM setting of about 1950. Those of skill in the art will understand that where higher shear is used, the amount of time needed to forming a uniform consistency can be shorter. Varying levels of shear can be used depending on the viscosity of the ingredients used in the premix and the other mixing conditions (temperature, volume of batches, mixing time, etc).

Examples of suitable methods of making the microdroplet have been described in U.S. Patent Publ. No. 2005/0032916 to Deckner at paragraphs 37-50 and Examples (describing a method of making a perfume oil emulsion), 2005/0031659 to Deckner at paragraph 33 and Examples (describing a method of making an emulsion with a concentrated internal oil phase), and 2005/0031568 to Deckner at paragraph 32 and Examples (describing a method of making a concentrated oil-in-water emulsion). Those of skill in the art will understand that although some of the examples disclosed in the above three publications are for other types of compositions, similar methods of forming the emulsion (described herein as the premix) can be used.

In one embodiment, the premix comprises about 70%, or about 80%, or above 93% of internal hydrophobic agent phase by weight of the premix.

### 4. Water Dispersible Surface Active Agent

Personal care compositions of the present invention contain one or more surface active agents. The water dispersible or water soluble surface active agent is preferably one that is capable of forming lather and may comprise a soap, an interrupted soap, a detergent, an anionic surfactant, a non-ionic surfactant or a mixture of one or more of these. The water dispersible surface active agent(s) can be present at a level of from about 2% to about 15%, preferably from about 3% to about 12%. In one embodiment, the amount of hydrophobic agent to water dispersible surface active has a weight ratio of 0.1:1 to about 10:1, or from about 0.5:1 to about 5:1, or from about 1:1 to about 3:1. Without intending to be bound by theory, it is believed that by providing the hydrophobic agent as a microdroplet, the composition can include a relatively high amount of the agent while being stable and providing desirable lubrication and shave related benefits.

Soaps may include, for example, the sodium, potassium and lower alkanolamine (preferably triethanolamine) salts of C12 22, preferably C14 18, fatty acids. Typical fatty acids include lauric, myristic, palmitic and stearic acid and mixtures thereof. The preferred fatty acids are palmitic and stearic. The interrupted soaps may include, for example, the sodium, potassium and lower alkanolamine (preferably triethanolamine) salts of N-fatty acyl sarcosines, wherein the fatty acyl moiety has 12 to 22, preferably 14 to 18, carbon atoms. Typical sarcosines include stearoyl sarcosine, myristoyl sarcosine, palmitoyl sarcosine, oleoyl sarcosine, lauroyl sarcosine, cocoyl sarcosine and mixtures thereof. The soaps and the interrupted soaps may be utilized in the preneutralized form (i.e., as the sodium, potassium or alkanolamine salt) or in the free acid form followed by subsequent neutralization with sodium hydroxide, potassium hydroxide and/or lower alkanolamine (preferably triethanolamine). In any event, the final composition preferably contains sufficient base to neutralize or partially neutralize the soap component and adjust the pH to the desired level (typically between 5 and 10, more typically between 6 and 9). It is most preferred that the shaving composition includes a soap (e.g., triethanolamine palmitate/stearate), an interrupted soap (e.g., triethanolamine stearoyl/myristoyl sarcosinate), or a mixture thereof.

The water dispersible surface active agent may also optionally include a non-ionic, amphoteric and/or anionic surfactant. Suitable non-ionic surfactants will typically have an HLB of 9 or more and include the polyoxyethylene ethers of fatty alcohols, acids and amides, particularly those having 10 to 20, preferably 12 to 18, carbon atoms in the fatty moiety and about 2 to 60, preferably 4 to 30, ethylene oxide units. These include, for example, Oleth 20, Steareth 21, Ceteth 20, Laureth 4 and Laureth 23. Other non-ionic surfactants include the polyoxyethylene ethers of alkyl substituted phenols, such as Nonoxynol-4 and Nonoxynol-20, fatty alkanolamides such as Lauramide DEA and Cocamide MEA, polyethoxylated sorbitan esters of fatty acids, such as Polysorbate 20, lauryl polyglucoside, sucrose ester fatty acids, sucrose laurate, and polyglycerol 8 oleate. Suitable amphoteric surfactants include, for example, the betaines and sultaines such as cocoamidopropyl betaine, coco dimethyl carboxymethyl betaine, lauroamphoacetate, cocaminopropionic acid, and mixtures thereof. Others include isononyl isononanoate, polyhydroxystearic acid, ethylhexyl isononanoate, sodium cocamidopropyl PG-Dimonium chloride, Cetearyl alcohol, cholesterol, and stearyl alcohol. Suitable anionic surfactants include, for example, the sodium, potassium, ammonium and substituted ammonium salts (such as the mono-, di- and triethanolamine salts) of C8 C22, preferably C12 C18, alkyl sulfates (e.g., sodium lauryl sulfate, ammonium lauryl sulfate), alkyl sulfonates (e.g., ammonium lauryl sulfonate), alkylbenzene sulfonates (e.g., ammonium xylene sulfonate), acyl isethionates (e.g., sodium cocoyl isethionate), acyl lactylates (e.g., sodium cocoyl lactylate), alkyl ether sulfates (e.g., ammonium laureth sulfate, ammonium laurylether sulfate), sodium methyl cocoyl taurate, sodium lauryl sulfoacetate, and dioctyl sodium sulfosuccinate.

In one embodiment, the composition is free or essentially free of soap. As used herein, “essentially free” of a component means that no amount of that component is deliberately incorporated into the composition. In one embodiment the composition is a self-foaming soap free shave gel as described in U.S. Pat. No. 5,500,211.

### 5. Lubricant

The lubricious water soluble polymer will generally have a molecular weight greater between about 300,000 and 15,000,000 daltons, preferably more than about one million daltons, and will include a sufficient number of hydrophilic moieties or substituents on the polymer chain to render the polymer water soluble. The polymer may be a homopolymer, copolymer or terpolymer. Examples of suitable lubricious water soluble polymers include polyethylene oxide, polyvinylpyrrolidone, and polyacrylamide. A preferred lubricious water soluble polymer comprises polyethylene oxide, and more particularly a polyethylene oxide with a molecular weight of about 0.5 to about 5 million daltons. Particularly suitable polyethylene oxides include, for example, PEG-14M (MW≅600,000) PEG-23M (MW≅1 million), PEG-45M (MW≅2 million) and PEG-90M (MW≅4 million). The lubricious water soluble polymer will generally be included in the post foaming gel composition in an amount of about 0.005% to about 3%, preferably about 0.01% to about 1%, by weight.

The water insoluble particles may include inorganic particles or organic polymer particles. Examples of inorganic particles include titanium dioxide, silicas, silicates and glass beads, with glass beads being preferred. Examples of organic polymer particles include polytetrafluoroethylene particles, polyethylene particles, polypropylene particles, polyurethane particles, polyamide particles, or mixtures of two or more of such particles. Any of the forgoing particles may also include a surface treatment to make the particles more readily dispersible or improve their cosmetic aesthetics. Preferred are polytetrafluoroethylene particles (e.g., PTFE particles available from MicroPowders, Inc. under the tradename Microslip). Preferably the water insoluble particles will have an average particle size of about 1 μm to about 100 μm, more preferably about 2 μm to about 50 μm, and most preferably about 5 μm to about 15 μm. The particles may be of any desired shape including spherical bead, elongated fiber or irregular shape, with spherical bead being the preferred shape. Generally the water insoluble particles will be included in the post foaming gel composition in an amount of about 0.01% to about 5%, preferably about 0.1% to about 2%, by weight.

The hydrogel-forming polymer is a highly hydrophilic polymer that, in water, forms organized three-dimensional domains of approximately nanometer scale. The hydrogel-forming polymer generally has a molecular weight greater than about one million daltons (although lower molecular weights are possible) and typically is at least partially or lightly crosslinked and may be at least partially water insoluble, but it also includes a sufficient number of hydrophilic moieties so as to enable the polymer to trap or bind a substantial amount of water within the polymer matrix and thereby form three-dimensional domains. It has been found that shave gel compositions that include the hydrogel-forming polymer have improved gel structure and reduced coefficient of friction (i.e., increased lubricity). Examples of suitable hydrogel-forming polymers include a polyacrylic acid or polymethacrylic acid partially esterified with a polyhydric alcohol; hydrophilic polyurethanes; lightly crosslinked polyethylene oxide; lightly crosslinked polyvinyl alcohol; lightly crosslinked polyacrylamide; hydrophobically modified hydroxyalkyl cellulose; hydroxyethyl methacrylate; and crosslinked hyaluronic acid. Generally, the hydrogel-forming polymer will be included in the post foaming gel composition in an amount of about 0.0005% to about 3%, preferably about 0.001% to about 0.5%, more preferably about 0.002% to about 0.1%, by weight.

A preferred hydrogel-forming polymer comprises polyacrylic acid partially esterified (e.g., about 40% to 60%, preferably about 50%, esterified) with glycerin. Such a polymer includes glyceryl acrylate/acrylic acid copolymer (MW>one million). It is believed that the glyceryl acrylate/acrylic acid copolymer forms a clathrate that holds water, which, upon release supplies lubrication and moisturization to the skin A preferred source of glyceryl acrylate/acrylic acid copolymer is available from ISP Technologies, Inc. (United Guardian Inc.) under the tradename Lubrajel®, particular the form known as Lubrajel® oil which contains about 1.0%-1.3% glyceryl acrylate/acrylic acid copolymer in aqueous glycerin (^{˜}40% glycerin). Lubrajel® oil also includes about 0.6% PVM/MA copolymer (also known as methoxyethylene/maleic anhydride copolymer), which may further contribute to the lubricity of this source. Most preferably, the post foaming gel composition will include about 0.25% to about 4% Lubrajel® oil in order to provide a preferred level of about 0.002% to about 0.05% of the glyceryl acrylate/acrylic acid copolymer. This amount of Lubrajel® oil will also provide about 0.001% to about 0.03% of PVM/MA copolymer.

### 6. Post Foaming Agent

The post-foaming agent, when included in the post foaming gel composition, may be any volatile hydrocarbon or halohydrocarbon with a sufficiently low boiling point that it will volatilize and foam the gel upon application to the skin, but not so low that it causes the gel to foam prematurely. The typical boiling point of such an agent generally falls within the range of −20° to 40° C. Preferred post-foaming agents are selected from saturated aliphatic hydrocarbons having 4 to 6 carbon atoms, such as n-pentane, isopentane, neopentane, n-butane, isobutane, and mixtures thereof. Most preferred is a mixture of isopentane and isobutane in a weight ratio (IP:IB) of about 1:1 to about 9:1, preferably about 2:1 to about 7:1, most preferably about 3:1. The post-foaming agent will normally be selected so as to provide a vapor pressure at 20° C. of about 3 to about 20 psig, preferably about 5 to about 15 psig. The post-foaming agent will be present in an amount to provide the post foaming gel composition with a sufficiently rapid turnover—that is, transition from gel to foam when contacted with the skin—typically, in about 2 to about 30 seconds, preferably in about 5 to about 15 seconds.

### 7. Carrier

The carrier is preferably dermatologically acceptable, meaning that the carrier is suitable for topical application to the keratinous tissue, has good aesthetic properties, is compatible with the actives of the present invention and any other components, and will not cause any safety or toxicity concerns. In one embodiment, the post foaming gel composition comprises from about 50% to about 99.99%, preferably from about 60% to about 93%, more preferably from about 70% to about 90%, and even more preferably from about 80% to about 85% of the carrier by weight of the composition. In one embodiment, the carrier comprises water.

### 8. Other Adjunct Ingredients

Although not necessary to forming a useful shave gel composition, other cosmetic ingredients may be advantageously added to improve the application aesthetics and/or achieve other shave benefits. For example, the composition may include one or more of the following components: beard wetting agents, skin conditioning agents (e.g., vitamins A, C and E, aloe, allantoin, panthenol, alpha-hydroxy acids, phospholipids, triglycerides, botanical oils, amino acids), foam boosters, emollients, humectants (e.g., glycerin, sorbitol, propylene glycol), fragrances, colorants, antioxidants, preservatives, organic cosolvens, etc. Nonlimiting examples of suitable organic cosolvents comprising at least one of glycerin, diglycerin, sorbitol, butylene glycol, propylene glycol, polyethylene glycol, and a mixture thereof. It is particularly preferred to include glycerin in the shave gel composition of the present invention, preferably in an amount of about 0.1% to about 3%, more preferably about 0.3% to about 1%, by weight. The organic cosolvent is believed to improve the emolliency of the composition.

It may be advantageous to include a sorbitan fatty ester or a sucrose fatty ester, typically in an amount of about 0.1% to about 3%, preferably about 0.3% to about 2%, by weight. These materials have multifunctional properties of emulsifier, moisturizer and anti-irritant. Sorbitan fatty esters include sorbitan stearate, sorbitan oleate, sorbitan isostearate, sorbitan laurate, sorbitan dioleate, etc. Sucrose fatty esters include sucrose stearate, sucrose oleate, sucrose isostearate, sucrose cocoate, sucrose distearate, etc. The sorbitan esters and sucrose esters may be mixtures of mono-, di- and tri-esters.

It may also be desirable to include an ester of a fatty acid, typically in an amount of about 0.5% to about 5%, preferably about 1% to about 4%, by weight. Useful fatty esters include glyceryl fatty esters such as, for example, glyceryl oleate and glyceryl dioleate, and fatty alcohol esters such as, for example, isostearyl linoleate, isocetyl oleate, and isostearyl isostearate. These materials provide emolliency, lubrication and gel structure.

It may further be desirable to include a propoxylated fatty amide, typically in an amount of about 0.5% to about 5%, preferably about 1% to about 3%, by weight. The propoxylated fatty amide will typically have from 1 to 3 propoxyl groups attached to a hydroxyloweralkyl fatty amide. Thus, suitable propoxylated fatty amides include, for example, PPG-2-hydroxyethyl coco/isostearamide, PPG-3-hydroxyethyl linoleamide, and PPG-2-hydroxyethyl cocamide.

The compositions of the present invention can comprise one or more thickening agents, preferably from about 0.05% to about 10%, more preferably from about 0.1% to about 5%, and even more preferably from about 0.25% to about 4%, by weight of the composition. Nonlimiting classes of thickening agents include those selected from the group consisting of: Carboxylic Acid Polymers (crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol); Crosslinked Polyacrylate Polymers (including both cationic and nonionic polymers, such as described in U.S. Pat. Nos. 5,100,660; 4,849,484; 4,835,206; 4,628,078; 4,599,379, and EP 228,868); Polymeric sulfonic acid (such as copolymers of acryloyldimethyltaurate and vinylpyrrolidone) and hydrophobically modified polymeric sulfonic acid (such as crosspolymers of acryloyldimethyltaurate and beheneth-25 methacrylate); Polyacrylamide Polymers (such as nonionic polyacrylamide polymers including substituted branched or unbranched polymers such as polyacrylamide and isoparaffin and laureth-7 and multi-block copolymers of acrylamides and substituted acrylamides with acrylic acids and substituted acrylic acids); Polysaccharides (nonlimiting examples of polysaccharide gelling agents include those selected from the group consisting of cellulose, carboxymethyl hydroxyethylcellulose (sold under the trademarks “Natrosol”), cellulose acetate propionate carboxylate, hydroxyethylcellulose, hydroxyethyl ethylcellulose, hydroxypropylcellulose (sold under the trademarks “Klucel”), hydroxypropyl methylcellulose, methyl hydroxyethylcellulose, microcrystalline cellulose, sodium cellulose sulfate, and mixtures thereof); Gums (i.e. gum agents such as acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof); and crystalline, hydroxyl-containing fatty acids, fatty esters or fatty waxes (such as microfibrous bacterial cellulose structurants as disclosed in U.S. Pat. Nos. 6,967,027 to Heux et al.; 5,207,826 to Westland et al.; 4,487,634 to Turbak et al.; 4,373,702 to Turbak et al. and 4,863,565 to Johnson et al., U.S. Patent Publ. No. 2007/0027108 to Yang et al.)

In one embodiment, the adjunct ingredient include one or more of the sensates or excipients suitable for use on skin. These sensates or excipients can be those which are commonly used in cosmetic and personal care compositions on the market today. Each of the additives can be provided at from about 0.001% to about 10%, or from about 0.1% to about 5% by weight of the composition. Non-limiting examples of suitable additives include one or more of: Bisabolol and Ginger root; sodium polyethylene glycol 7 olive oil carboxylate; Lauryl p-Cresol Ketoxime, 4-(1-Phenylethyl)1,3-benzenediol, Lupin (Lupinus albus) oil & wheat (Triticum vulgare) germ oil unsaponifiables, Hydrolyzed lupin protein, Extract of L-lysine and L-arginine peptides, Oil soluble vitamin C, Evodia rutaecarpa fruit extract, Zinc pidolate and zinc PCA, Alpha-linoleic acid, p-thymol, extract of camellia sinensis (such as white tea extract); panthenol; glycyrrhizinate salts, and combinations thereof; and skin and/or hair care active selected from the group consisting of sugar amines, vitamin B₃, retinoids, hydroquinone, peptides, farnesol, phytosterol, dialkanoyl hydroxyproline, hexamidine, salicylic acid, N-acyl amino acid compounds, sunscreen actives, water soluble vitamins, oil soluble vitamins, hesperedin, mustard seed extract, glycyrrhizic acid, glycyrrhetinic acid, carnosine, Butylated Hydroxytoluene (BHT) and Butylated Hydroxyanisole (BHA), menthyl anthranilate, cetyl pyridinium chloride, tetrahydrocurmin, vanillin or its derivatives, ergothioneine, melanostatine, sterol esters, idebenone, dehydroacetic acid, Licohalcone A, creatine, creatinine, feverfew extract, yeast extract (e.g., Pitera®), beta glucans, alpha glucans, diethylhexyl syringylidene malonate, erythritol, p-cymen-7-ol, benzyl phenylacetate, 4-(4-methoxyphenyl)butan-2-one, ethoxyquin, tannic acid, gallic acid, octadecenedioic acid, p-cymen-5-ol, methyl sulfonyl methane, an avenathramide compound, fatty acids (especially poly-unsaturated fatty acids), anti-fungal agents, thiol compounds (e.g., N-acetyl cysteine, glutathione, thioglycolate), other vitamins (vitamin B 12), beta-carotene, ubiquinone, amino acids, their salts, their derivatives, their precursors, and/or combinations thereof; and a dermatologically acceptable carrier. These and other potentially suitable actives are described in greater detail in U.S. Patent Publication No. 2008/0069784 and 61/364,932 and U.S. Ser. No. 12/984,958. In another embodiment, the personal care composition further comprising a sensate. A non-limiting example of a suitable sensates is methyl naphthalenyl ketone. In one embodiment the composition comprises from about 0.001% to about 1% of methyl naphthalenyl ketone. The methyl naphthalenyl ketone can be a 1-(1,2,3,4,5,6,7,8-octahydro-2,3,8,8-tetramethyl-2naphthalenyl)-ethan-1-one molecule or an isomer or derivative thereof. Commercially available as Iso-E-Super from IFF of New York.

In yet another embodiment, the personal care composition further comprising from about 0.001% to about 1%, preferably from about 0.05% to about 0.5% of a cooling agent. Preferred cooling agents but not limited to are menthol, CoolAct 10, menthyl lactate, and combinations thereof.

The CTFA Cosmetic Ingredient Handbook, Second Edition (1992) describes a wide variety of nonlimiting cosmetic and pharmaceutical ingredients commonly used in the skin care industry, which are suitable for use in the compositions of the present invention. Examples of these ingredient classes include: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, fatty alcohols and fatty acids, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents, skin-conditioning agents, skin soothing and/or healing agents and derivatives, skin treating agents, thickeners, and vitamins and derivatives thereof. Additional non-limiting examples of additional suitable skin treatment actives are included in U.S. 2003/0082219 in Section I (i.e. hexamidine, zinc oxide, and niacinamide); U.S. Pat. No. 5,665,339 at Section D (i.e. coolants, skin conditioning agents, sunscreens and pigments, and medicaments); and US 2005/0019356 (i.e. desquamation actives, anti-acne actives, chelators, flavonoids, and antimicrobial and antifungal actives). Examples of suitable emulsifiers and surfactants can be found in, for example, U.S. Pat. No. 3,755,560, U.S. Pat. No. 4,421,769, and McCutcheon's Detergents and Emulsifiers, North American Edition, pages 317-324 (1986). It should be noted, however, that many materials may provide more than one benefit, or operate via more than one mode of action. Therefore, classifications herein are made for the sake of convenience and are not intended to limit the active to that particular application or applications listed. Other useful optional ingredients include: Anti-Wrinkle Actives and/or Anti-Atrophy Actives; Anti-Oxidants and/or Racial Scavengers; Anti-Inflammatory Agents; Anti-Cellulite Agents; Tanning Actives; Skin Lightening Agents; Sunscreen Actives; Water Soluble Vitamins; particulates; and combinations thereof.

The composition can also include other commonly included ingredients which are used in commercially available post foaming shave gels such as those described in US Patent Publ. Nos. 2006/0257349, 2006/0257350 and 2005/0175575.

The personal care composition of the present invention may also contain a hydrophobically modified cationic polysaccharide, modified with a hydrophobic substituent and a cationic substituent. The hydrophobically modified cationic polysaccharide is used at a level of from about 0.005% to about 3%, or from about 0.01% to about 2.0%, or from about 0.02 to about 1%, or from about 0.025% to about 0.5%, by weight. Non-limiting examples of suitable hydrophobically modified cationic polysaccharides comprise cellulose, starch and guar derivatives, particularly a derivatized hydroxyethyl cellulose ether (such as those sold under the Trade Name of SoftCAT™). Nonlimiting examples of hydrophobically modified quaternized hydroxyethyl cellulose ethers include: those referred to in US 2007 0031362 A1 from Union Carbide, and can be referred to by those skilled in the art as SoftCAT.

In yet another embodiment, the personal care compositions of the present invention contain a film forming system. The film forming system can be made up of at least one film forming material. In certain embodiments, it may be beneficial for more than one film forming material to make up the film forming system. Useful film forming materials include, but are not limited to, polyvinylpyrrolidone, polyethylene oxide, hydroxyethylcellulose, hydroxylpropylcellulose, starch, polyvinyl alcohol, albumins, cationic celluloses, xanthan, carageenan, sodium polystyrene sulfonate, sodium silicone t-butyl acrylate, sodium poly (styrene sulfonate/maleic anhydride), sodium poly (styrene sulfonate co acrylate), polyvinylsulfonate, polyvinyl sulfate, polyphosphate, polymethacrylate, sodium dextran sulphate, poly (ethylene oxide co styrene sulfonate), methylcellulose, hydroxypropylmethylcellulose, ethylhydroxyethylcellulose, methylhydroxyethylcellulose, agar, dextran, amphomer, celquat, glucamate DOE-120, Glucamate LT, polyquaterniums (e.g., PQ 2, 7, 10, 16, 17, 18, 19, 24, 27, and 46), Merquats, Quaternized PVP, proteins and polypeptides (e.g., collagen, elastin, keratin, and their quaternary derivatives such as CROQUAT and QUAT-Coll), adipic acid/dimethylaminohydroxypropyl/diethylenetriamine copolymer), PVP/methacrylate, Aquaflex (polyimide-1), Gantrez (copolymers of methyl vinyl ether and maleic anhydride) Styleze (vinyl pyrrolidone/acrylate/lauryl methacrylate copolymer), pectin, and mixtures thereof. Other film forming polymers are disclosed in US 2010/021409 at paragraphs 15-21.

Exemplary shaving composition embodiments provided by the present invention include the following concentration levels of film forming materials and surface active agents: from about 0.6% wt % to about 1.2 wt % of film forming materials and from about 20 wt % to about 30 wt % of surface active agents; from about 0.6% wt % to about 1.2 wt % of film forming materials and from about 10 wt % to about 20 wt % of surface active agents; from about 1.2% wt % to about 2 wt % of film forming materials and from about 20 wt % to about 30 wt % of surface active agents; and from about 0.6% wt % to about 2 wt % of film forming materials and from about 3 wt % to about 10 wt % of surface active agents.

### 9. Product Forms and Uses

The personal care compositions of the present invention can be used for as a hair removal preparation such as a post foaming shave gel. The present composition may be formulated as an aerosol foam or a post-foaming gel (which is the preferred form). It may be packaged in any suitable dispenser normally used for dispensing personal care compositions (such as shaving compositions). These include collapsible tubes, pump or squeeze containers, and aerosol-type dispensers, particularly those with a barrier to separate the post foaming gel composition from the propellant required for expulsion. In one embodiment, the composition is contained in a single chamber, meaning that the hydrophobic agent and the other ingredients are not physically separated on the shelf. In one embodiment, all components of the compositions can be present in a single chamber. Multiple chambers can also be present within the composition. The chambers can all house the same composition, or different compositions.

The latter type of dispensers include: (1) mechanically pressurized bag-in-sleeve systems in which a thin-walled inner bag containing the product is surrounded by an outer elastic sleeve that is expanded during the product filling process and provides dispensing power to expel the product (e.g., the ATMOS System available commercially from the Exxel Container Co.); (2) (a) a container preform comprising a polymeric preform and an elastically deformable band surrounding at least a portion of the polymeric perform such as described in U.S. 2009/0263174 to Chan et al; (3) manually activated air pump spray devices in which a pump system is integrated into the container to allow the user to pressurize the container with air in order to expel the product (e.g., the “AIRSPRAY” system available from Airspray International); (4) piston barrier systems in which the product is separated from the driving means by a tight-fitting piston which seals to the side of the container and may be driven by a spring under tension, by a vacuum on the product side of the piston, by finger pressure, by gas pressure to the piston, or by a variety of other means known to the packaging industry; and (5) bag-in-can (SEPRO) systems in which the product is contained in a flexible bag within a can, with a suitable propellant injected into the space between the can and the flexible bag. It is preferred to protect the composition from oxidation and heavy metal contamination. This can be achieved, for example, by purging the composition and container with nitrogen to remove oxygen and by utilizing inert containers (e.g., plastic bottles or bags, aluminum cans or polymer coated or lined cans).

The present composition can be used in combination with various hair removal applications (prior to, concurrently with, and/or after), including but not limited to shaving (wet or dry shaving, via electric razors, via powered or manual razors which can be reuseable or disposable, and combinations thereof), epilation, electrolysis, wax or depilatories as well as energy delivery devices to help regulate hair growth. Nonlimiting examples of energy deliver devices include: light, heat, sound (including ultrasonic waves and radio frequency), electrical energy, magnetic energy, electromagnetic energy (including radiofrequency waves and microwaves), and combinations thereof. The light energy may be delivered by devices including, but not limited to, lasers, diode lasers, diode laser bars, diode laser arrays, flash lamps, intense pulsed light (IPL) sources, and combinations thereof. See e.g. US2006/0235370A1.

### 10. In Shave Lubrication Test

It has been found that the personal care composition of the present invention provides for an in shave lubrication benefit as shown by reduced friction as measured by the In Shave Lubrication “ISL” Test defined herein. Reducing friction is important during the shave because a high friction skin surface results in bulging of the skin. When the skin bulges, the blade is more likely to engage the skin, increasing the chance for skin irritation. Therefore, by reducing friction the product helps protect the skin. In addition, lower friction results in less drag on the skin, which can also be a potential source of irritation. This method enables measurement of the coefficient of friction (CoF) of a shave preparation.

In Shave Lubrication Test Method: An apparatus designed to simulate lubrication during the shaving process is connected to an instrument capable of measuring frictional forces (for example, an Instron-type instrument) and containing a load cell of about 1 kg to about 100 kg. The rinsing apparatus comprises: 1) an air-activated clamping device capable of opening and closing to deliver pressures of about 10 psi to about 70 psi to simulate the pressure exerted by hands on hair during rinsing 2) keratinous tissue models as described herein affixed to two opposing sides of the clamping device and 3) one or more spray nozzles capable of delivering water flow rates of from about 50 ml/min. to about 1000 mL/min., for simulating shower conditions.

Procedure: Attach the rinsing apparatus to the base of a Stable Micro Systems TA XT Plus™ Texture Analyzer (TA) equipped with a 30 kg load cell, centering or aligning the clamps perpendicular to the load cell. Adjust water flow rate to approximately 200 ml/min and the water temperature to 103° F.+/−2° F. Set the air pressure for the TA clamps to approximately 30 psi. Set the instrument measurement settings as follows: TA settings, tension compression, test speed-10.0 mm/sec for 130 mm long pull. Set the macro for a total of 10 strokes. Run the first five strokes without the water on, then manually turn on the water for 2 min 15 sec for the second five strokes. During the test, data (g of force) will only be collected during the upward pull of the treated KTM, not on the return. Cover the pads on both the front and back side of the piston with a polyurethane skin pad (see JP2006233367 for details).

Wet a 2 inch by 9 inch piece of nonwoven KTM under hot (˜103° F.+/−2° F.) tap water for 30 seconds. Place 2 g+/−0.1 g of aerosol shave gel or 1 g+/−0.1 g of aerosol shave foam onto the nonwoven KTM and gently lather and spread by hand for 30 seconds. Rub excess foam on the back of the nonwoven KTM. Load the nonwoven KTM into the TA and start the test macro. At the end of the fifth stroke, turn on the rinse water. Initiate a test sequence which 1) instructs the instrument to raise the load cell to which the KTM is attached, at a rate of about 10 mm/sec 2) opens the clamps, and 3) instructs the instrument to lower the load cell. Repeat this sequence until a predetermined number of sequences may be executed, for example, 10. Between each sample, wipe the polyurethane skin pads with a piece of nonwoven KTM and an alcohol wipe to remove any possible build-up from the previous test. By calculating the total friction in grams of force (or other suitable unit of force) for dry friction and rinse friction, products may be ranked relative to each other to assess which products would be expected to have the most pleasant feel.

“KTM” as defined herein means a “Keratinous tissue mimic” which refers to one or more artificial substrates which may have one or more physical properties representative of keratinous tissue. The KTM used for the purposes of this application is TENCEL from Lenzing, Inc. Additional details on other KTMs is disclosed in Section I of U.S. Ser. No. 61/239,908 to Battaglia et al, filed Sep. 4, 2009.

### 11. Process of Making the Aerosol Shave Composition

One embodiment of the present invention provides for a process of making an aerosol shave composition comprising the steps of: forming a microdroplet premix comprising at least about 50% of a hydrophobic agent and up to about 50% of a carrier comprising water; and mixing said microdroplet premix with a second feed stream comprising: water dispersible surface active agent, a volatile post-foaming agent, and a carrier comprising water. In one embodiment, the step of forming said microdroplet premix comprises the subjecting the premix ingredients under a sufficiently high shear to achieve the microdroplets described herein. Examples of suitable methods for forming the microdroplet premix are described in U.S. Patent Publs. 2005/0031659, 2005/0031568, and 2005/0032916, each to Deckner, wherein the discontinuous oil phases is formed by said hydrophobic agent. In one embodiment, the step forming said microdroplet premix can be performed with a Speed Mixer DAC 800 FVZ with an RPM setting of about 1950. The mixing can be performed at room temperature but elevated or colder temperatures are also suitable.

The microdroplet premix is then added to the other aerosol shave composition ingredients, followed by mixing then addition of the propellant (i.e., the post foaming agent) then setting of the mixture to allow the aerosol shave composition to thicken and settle.

### 12. Examples

The following examples are formulated as described below. QS means quantity sufficient to reach 100%. All values are percent by weight.

One example of an aerosol shave composition in accordance with the present invention includes the following ingredients at the specified amounts by weight:
Water 35-90%
hydrophobic agent (i.e., dimethicone) 1-10%
Emulsifier (i.e., decyl glucoside) 0.1-5%
Water dispersible surface active agent (i.e., triethanolamine palmitate/stearate) 3-30%
Volatile post foaming agent (i.e., volatile hydrocarbon, carbon dioxide, nitrogen) 0-4%
Polymer (i.e., polyethylene oxide, polyvinylpyrrolidone) 0.04-0.25%
Thickener/Stabilizer (i.e., hydroxyethylcellulose, cationic soft cellulose) 0.1-0.75
Others, Organic sosolvents, etc (i.e., emulsifiers, polar alcohols) 0.0%-0.30% One specific example can be made with the following formula:

| TRADE OR | | |
|---|---|---|
| COMMON NAME | CTFA NAME | As Added |
| Dow Corining 200 | Dimethicone | 0.00-2.475% |
| Fluid 30,000 cst. | | |
| Plantaren 1200 N UP | Lauryl Glucoside | 0.00-0.275% |
| Water | Water | 76.1423-76.9423% |
| Edenor C16 92 MY | Palmitic Acid | 7.7500% |
| Triethanolamine | Triethanolamine | 6.0500% |
| Emersol 132 | Stearic Acid | 2.6000% |
| Monomuls 90-018 | Glyceryl Oleate | 2.0000% |
| Sorbitol 70% | Sorbitol | 1.0000% |
| Natrosol 250 HHR | Hydroxyethyl- | 0.5000% |
| | cellulose | |
| Menthol, Natural | Menthol | 0.00-0.15% |
| Polyox WSR-301 | PEG-90M | 0.1700% |
| Microslip 519 | PTFE | 0.1500% |
| Polyox WSR N-12K | PEG-23M | 0.0500% |
| Propylene Glycol | Propylene Glycol | 0.0240% |
| TBP-4 Blowing Agent | Isopentane and | 2.85% |
| | Isobutane | |
| Adjuncts (colorants, | | Remainder to 100% |
| fragrances, etc) | | |

Personal care compositions in accordance with the present invention provide better lubrication than similar compositions without hydrophobic agents. The increased lubrication can be shown by a decrease of about 5% to about 50%, or from about 20% to about 40%, or about 30%, in drag force compared to samples without the hydrophobic agent in the form of a microdroplet.

Examples of hydrophobic agents in the form of a microdroplet:

### Example A

Step 1: Forming the premix

A 50/50 mixture of surfactant (such as Plantaren 1200 N UP) is added to an organic solvent (such as glycerin) to form the carrier. The hydrophobic agent is then added in small batches to carrier while stirring by hand with a spatula or being mixed by a Kitchen Aid® Ultra Power Mixer at any setting between 2 and 4 until a uniform consistency is observed and a microdroplet is formed. In this example, the hydrophobic agent is Dow Corning Xiameter 300,cs (dimethicone). The weight ratio of carrier to hydrophobic agent is from 1:1 to 1:20, or from 1:9.

Step 2: Adding the premix to other components to form the personal care composition can be done by conventional means of making shave preparations, i.e. using a standard bench or lab mixer such as Cafarmo Stirrer, Model BDC1850 at 350 RPM for about 30 minutes or until a substantially homogenous mixture is obtained. Larger or smaller batch sizes can use different RPM settings.

### Example B

In this example, a commercially available hydrophobic agent can be used which is believed to already be in microdroplet form.

Step 1: Obtain a pre-made hydrophobic agent premix in microdroplet form (such as Dow Corning Xiameter MEM-1664 Emulsion (50% dimethicone).

Step 2: Add the hydrophobic agent in small batches into a mixing chamber with the other components of the personal care composition. Mixing is performed using a Cafarmo Stirrer, Model BDC1850 at 350 RPM at room temperature for about 30 minutes, or until a substantially homogeneous mixture is formed. Repeat step 2 adding small batches of the premade hydrophobic agent premix each time (such as from about ⅕^{th} to about 1/30^{th} or from about 1/10^{th} to about 1/20^{th} of the total amount of the hydrophobic agent premix). The amount of hydrophobic agent premix can be 2.75% by weight.

### Example C

### Premix with an Cooling Additive

Step 1: Mixing the hydrophobic agent and Additive

Combining 60.8 grams of a hydrophobic agent such as DC Xiameter 30,000 cs (dimethicone) with 0.1 grams of an additive such as menthol and/or fragrance. This mixing is performed while being mixed by hand using a spatula until the menthol is completely dissolved.

Step 2: Forming the premix

Combine the mixture from step 1) with 9.1 grams of a surfactant such as Plantaren 1200 N UP. The hydrophobic agent is added in small batches to the surfactant while being mixed by hand with a spatula until a substantially homogenous mixture is formed. Additional small batches are added and mixed until a substantially homogenous mixture is formed.

Step 3: Adding the premix to other components to form the personal care composition can be done by conventional means of making shave preparations, i.e. using a standard bench or lab mixer such as Cafarmo Stirrer, Model BDC1850 at 350 RPM for about 15 minutes or until a substantially homogenous mixture is obtained.

### Example D

An example of the “other components” suitable for use in Examples A-C.

Step 1: Blue dye is formed by mixing 15.8 grams of distilled water with 4.0 grams of PEG and 0.2 grams of FD&C Blue dye #1, by hand mixing until dissolution.

Step 2: 1528.7 grams of distilled water is added to 19.4 grams of sorbitol (70% in water) and mixed with a Cafarmo Stirrer, Model BDC1850 at 100 RPM.

Step 3: a combination of 9.72 grams of Natrosol 250 HHR (Hydroxyethylcellulose), 3.30 grams of Polyox WSR-301 (PEG 90M), 0.97 grams of Polyox WSR N-12K (PEG 23M), and 2.91 grams of Microslip 519 are sloly added to the mixture of Step 2 and heated to 80 C and mixed for 30 minutes or until a substantially homogenous mixture is observed.

Step 4: At 80 C, 150.6 grams palmitic acid C16 (95%), 50.5 grams of stearic acid (Emersol 132), and 38.9 grams of Monomuls 90-018 (Glyceryl Oleate) are added and mixed for about 30 minutes or until a substantially homogenous mixture is observed.

This mixture can be added to the premix at a weight ratio of 97.25:2.75, or 97.15:2.85 (such as for Example C).

### Example E

An example of the “other components” suitable for use in Examples A-C.

Step 1: 11.76 grams of triethanolamine at 99% can be mixed with a Cafarmo Stirrer, Model BDC1850 at 350 RPM with a beater impeller in a heated water bath of 80 C. RPM is adjusted so aeration does not occur. Mixing can be done for 30 minutes or until a substantially homogenous mixture is observed.

Step 2: The heated water bath is replaced with cool water to allow the mixture to cool to about 4° C.

Step 3: Fragrance ingredient at a amount of 16.5 grams and colorant such as FD&C blue dye #1 at a 1% solution at 3.886 grams are added to the triethanolamine and further mixed for 10 minutes or until a substantially homogenous mixture is observed.

This mixture can be added to the premix at a weight ratio of 97.25:2.75, or 97.15:2.85 (such as for Example C).

It should be understood that every maximum numerical limitation given throughout this specification includes every lower numerical limitation, as if such lower numerical limitations were expressly written herein. Every minimum numerical limitation given throughout this specification includes every higher numerical limitation, as if such higher numerical limitations were expressly written herein. Every numerical range given throughout this specification includes every narrower numerical range that falls within such broader numerical range, as if such narrower numerical ranges were all expressly written herein.

All parts, ratios, and percentages herein, in the Specification, Examples, and Claims, are by weight and all numerical limits are used with the normal degree of accuracy afforded by the art, unless otherwise specified.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”

All documents cited in the DETAILED DESCRIPTION OF THE INVENTION are, in the relevant part, incorporated herein by reference; the citation of any document is not to be construed as an admission that it is prior art with respect to the present invention. To the extent that any meaning or definition of a term or in this written document conflicts with any meaning or definition in a document incorporated by reference, the meaning or definition assigned to the term in this written document shall govern.

Except as otherwise noted, the articles “a,” “an,” and “the” mean “one or more.”

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An aerosol shave composition comprising:
a. from about 2% to about 25% of a water dispersible surface active agent;
b. from about 40% to about 95% of a carrier comprising water;
c. from about 1% to about 6% of a volatile post-foaming agent; and
d. from about 0.01 to about 15% of a hydrophobic agent, wherein said hydrophobic agent forms at least one microdroplet having a particle size from about 0.15 microns to about 10 microns, wherein the composition is substantially homogenous.

2. The aerosol shave composition of claim 1, wherein said at least one microdroplet has a particle size of from about 0.5 microns to about 5 microns.

3. The aerosol shave composition of claim 1, wherein said hydrophobic agent forms a plurality of microdroplets comprising an average particle size of from about 0.5 microns to about 3 microns.

4. The aerosol shave composition of claim 3, wherein at least one of said plurality of microdroplets has a particle size of from about 0.5 microns to about 5 microns.

5. The aerosol shave composition of claim 1, wherein said aerosol shave composition is a post foaming gel.

6. The aerosol shave composition of claim 1, wherein said hydrophobic agent comprises at least one of a silicon polymer, a mineral oil, vitamin E, vitamin A, anhydrous polyols, and mixtures thereof.

7. The aerosol shave composition of claim 6, wherein said silicon polymer comprises at least one of an organosiloxane, an amino-functional siloxane, and combinations thereof.

8. The aerosol shave composition of claim 7, wherein said organosiloxane comprises at least one of a dimethicone, a trimethylsiloxane, a polydimethylsiloxane, a silicone elastomer, and combinations thereof.

9. The aerosol shave composition of claim 1, further comprising from about 0.005% to about 3% of a hydrophobically modified cationic polysaccharide, modified with a hydrophobic substituent and a cationic substituent.

10. The aerosol shave composition of claim 1, further comprising a lubricant comprises at least one of: a lubricious water soluble polymer, a water insoluble particle, a hydrogel-forming polymer, and a mixture thereof.

11. The aerosol shave composition of claim 1, wherein the lubricant comprises at least one of: from about 0.01% to about 1%, of a lubricious water soluble polymer, from about 0.01% to about 5% of a water insoluble particle, from about 0.0005% to about 3%, of a hydrogel-forming polymer, and a mixture thereof, by weight of the post foaming gel composition.

12. The aerosol shave composition of claim 11, wherein said lubricious water soluble polymers comprises at least one of a polyethylene oxide, a polyvinylpyrrolidone, a polyacrylamide, and a mixture thereof.

13. The aerosol shave composition of claim 11, wherein said water insoluble particles comprises at least one of an inorganic particles, an organic polymer particles, and a mixture thereof.

14. The aerosol shave composition of claim 11, wherein said hydrogel-forming polymers comprises at least one of: a polyacrylic acid or polymethacrylic acid partially esterified with a polyhydric alcohol; a hydrophilic polyurethanes; a lightly crosslinked polyethylene oxide; a lightly crosslinked polyvinyl alcohol; a lightly crosslinked polyacrylamide; a hydrophobically modified hydroxyalkyl cellulose; a hydroxyethyl methacrylate; and crosslinked hyaluronic acid.

15. The aerosol shave composition of claim 14, wherein said hydrogel-forming polymer comprises polyacrylic acid partially esterified with glycerin.

16. The aerosol shave composition of claim 1, wherein said water dispersable surface active agent comprises an anionic surfactant.

17. The aerosol shave composition of claim 1, wherein said water dispersible surface active agent is free or essentially free of a soap, and wherein said water dispersible surface active agent lathering surfactants comprises a non-ionic surfactant.

18. The aerosol shave composition of claim 1, wherein the hydrophobic agent comprises a silicone having a viscosity of from about 30,000 to about 60,000 centistokes, at 25° C.

19. The aerosol shave composition of claim 1, further comprising an organic cosolvent comprising at least one of water, glycerin, diglycerin, sorbitol, butylene glycol, propylene glycol, polyethylene glycol, and a mixture thereof.

20. The aerosol shaving composition of claim 1, wherein the composition is contained in a single chamber of a container.
